(19) United States
(12) Patent Application Publication
(10) Pub. No.: US 2016/0113645 A1
(43) Pub. Date: Apr. 28, 2016

(54) IMPLANT WITH SUTURE ANCHOR FIXATION CAPABILITY

(71) Applicant: Zimmer, Inc., Warsaw, IN (US)

(72) Inventors: Philippe Hardy, Paris (FR); Guido Marra, Downers Grove, IL (US); Steven Humphrey, Warsaw, IN (US); Terry W. Wagner, Mishawaka, IN (US)

(21) Appl. No.: 14/867,134

(22) Filed: Sep. 28, 2015

Related U.S. Application Data
(60) Provisional application No. 62/069,043, filed on Oct. 27, 2014.

(51) Int. Cl. A61B 17/04; A61F 2/40

(52) U.S. Cl. CPC A61B 17/0401; A61F 2/4059

(57) ABSTRACT

An implantable device and various systems and methods related to the device are disclosed. The implantable device has a bone implant component configured to be secured to at least one bone of a patient with at least one recess therein. The bone implant component is configured to allow a suture anchor to be disposed within the recess and has a restriction therein that is engageable by the suture anchor to retain the suture anchor within the bone implant component. Methods for implanting a suture anchor in the implantable device are also disclosed.

### CLAIM OF PRIORITY

This application claims the benefit of U.S. Provisional Patent Application Ser. No. 62/069,043, filed on Oct. 27, 2014, the benefit of priority of which is claimed hereby, and which is incorporated by reference herein in its entirety.

### TECHNICAL FIELD

The present patent application relates to surgical implants, and more particularly, to apparatuses and methods related to surgical implants that accommodate and retain suture anchors therein.

### BACKGROUND

Surgical implants are utilized to treat a wide range of bodily injuries, maladies, and conditions. For example, orthopedic implants are used to replace or augment body components or portions of body components that cannot be regenerated or are no longer functioning properly. Orthopedic implants include spinal implants, dental implants, artificial shoulders, knees, hips, and ankle joints. Various trauma related implants can be utilized to help stabilize and treat a patient. Examples of trauma related implants include screws, nails, fixation systems, plate systems, etc.

Orthopedic implants have been developed for the shoulder joint. In a healthy shoulder, the proximal humerus is generally ball-shaped, and articulates within a socket formed by the scapula, called the glenoid, to form the shoulder joint. Implant systems for the total replacement of the shoulder joint, i.e., a total shoulder arthroplasty, generally replicate the natural anatomy of the shoulder, and typically include a humeral component having a stem which fits within the humeral canal, and an articulating head which articulates within the socket of a glenoid component implanted within the glenoid of the scapula. Reverse type implant systems have been developed in which the conventional ball-and-socket configuration that replicates the natural anatomy of the shoulder is reversed, such that a concave recessed articulating component is provided at the proximal end of the humeral component that articulates against a convex portion of the glenoid component.

### Overview

Various procedures may be performed to repair soft tissue and bone in the body. These can be performed concurrently with the implantation of various devices. In some cases, it is desirable to repair the soft tissue by affixing the soft tissue to an area on the bone by providing a suture through a selected portion of the soft tissue while securing the other end of the suture to the selected area on the bone using a suture anchor.

The present inventors have recognized that traditional suture holes and features provided in some implantable devices for reattaching soft tissue, stabilizing bone fragments, or securing bone structure can sometimes be difficult and time consuming to access in the surgical field and can be at risk for failure due to corners, abrasion, etc. In view of these concerns, the present inventors have recognized that various implantable devices can be configured to allow one or more suture anchors to be affixed therein. Thus, the present inventors have recognized that implantable devices can be provided with one or more features such as a recess that is adapted to receive and retain a suture anchor therein. Implantable devices with suture anchor fixation capability are described herein and can be used, for example, in various orthopedic devices (including artificial hips, knees, ankles, elbows, and shoulders), trauma devices (including bone plates, intramedullary rods, etc.) and oncology and/or reconstructive systems (including limb salvage systems, severe bone loss systems, etc.).

To better illustrate the devices, systems, and methods disclosed herein, a non-limiting list of examples is provided here:

In Example 1, an implantable device can include a bone implantation component. The bone implant component can be configured to be secured to at least one bone of a patient and can have at least one recess therein. The bone implant component can be configured to allow a suture anchor to be disposed within the recess and can have a restriction therein that is engageable by the suture anchor to retain the suture anchor within the bone implant component.

In Example 2, the device of Example 1 wherein the recess can comprise a hole formed by a first section with a first diameter and a second section having an enlarged diameter with respect to the first diameter of the first section, and the first section can act as the restriction to retain the suture anchor.

In Example 3, the device of any of Examples 1 to 2 wherein the first section can include a transition surface between the first section and the second section, the transition surface can act as a shoulder to retain the suture anchor against the first section and substantially within the second section of the bone implant component.

In Example 4, the device of any of Examples 1 to 3 wherein the hole can extend through the bone implant component such that the first section can be accessed from either a first or second side of the bone implant component.

In Example 5, the device of any of Examples 1 to 4 wherein the bone implant component can have a bi-symmetrical configuration such that the hole includes the first section, the second section, and a third section, and wherein the third section can have a diameter that is substantially the same as the diameter of the second section.

In Example 6, the device of any of Examples 1 to 5 wherein the first section can comprise a suture anchor retention feature and the second section comprises a suture anchor access feature.

In Example 7, the device of any of Examples 1 to 6 wherein a wall of the first section can have a thread configured to mate with a thread of the suture anchor.

In Example 8, the device of any of Examples 1 to 7 wherein the bone implant component can comprise a humeral component for use in a shoulder arthroplasty.

In Example 9, the device of any of Examples 1 to 8 wherein the bone implant component can further comprise a coupling of the humeral component that includes the at least one recess.

In Example 10, a surgical method can comprise securing a bone implant component to at least one bone of a patient, the bone implant component can have at least one recess therein, positioning a suture anchor within the recess, and actuating the suture anchor to engage a wall of the recess to secure the suture anchor within the bone implant component.

In Example 11, the method of Example 10 wherein actuating the suture anchor can comprise inserting the suture anchor in a temporary shape through a first section to a second section of the recess, wherein the first section can have a reduced diameter with respect to a diameter of the second section, and bringing the suture anchor into contact with at least a portion of the first section.

In Example 12, the method of any of Examples 10 to 11 wherein the portion of the first section can comprise a transition surface between the first section and the second section, the transition surface can act as a shoulder to retain the suture anchor against the first section and substantially within the second section of the bone implant component.

In Example 13, the method of any of Examples 10 to 12 wherein actuating the suture anchor can comprise threading the suture anchor into a mating thread in the wall of the recess.

In Example 14, the method of any of Examples 10 to 13 wherein the bone implant component can comprise a humeral component for use in a shoulder arthroplasty.

In Example 15, a system can comprise a shoulder implant and a suture anchor. The shoulder implant can have a humeral component configured to be secured to at least one bone of a patient and having at least one recess with a restriction therealong. The suture anchor can be engageable with the restriction to retain a suture anchor within the humeral component.

In Example 16, the system of Example 15 wherein the recess can comprise a hole formed by a first section with a first diameter and a second section having an enlarged diameter with respect to the first diameter of the first section, and wherein the first section can comprise the restriction.

In Example 17, the system of any of Examples 15 to 16 wherein the first section can include a transition surface between the first section and the second section. The transition surface can act as a shoulder to retain the suture anchor against the first section and substantially within the second section of the humeral component.

In Example 18, the system of any of Examples 15 to 17 wherein the hole can extend through the humeral component such that the first section can be accessed from either a first or second side of the humeral component.

In Example 19, the system of any of Examples 15 to 18 wherein the humeral component can have a bi-symmetrical configuration and can comprise the first section, the second section, and a third section, and wherein the third section can have a diameter that is substantially the same as the diameter of the second section.

In Example 20, the system of any of Examples 15 to 19 wherein a wall of the restriction can have a thread configured to mate with a thread of the suture anchor.

In Example 21, the apparatus, system, or method of any one or any combination of Examples 1-20 can optionally be configured such that all elements or options recited are available to use or select from.

These and other examples and features of the present devices, systems, and methods will be set forth in part in the following Detailed Description. This overview is intended to provide a summary of subject matter of the present patent application. It is not intended to provide an exclusive or exhaustive removal of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an implantable device that can be used in a total shoulder arthroplasty or a hemi shoulder arthroplasty, including a humeral component and a glenoid component, according to an example of the present application.

FIG. 2 is a plan view of the coupling having suture anchor holes therein according to an example of the present application.

FIG. 3 is a cross-sectional view taken along line of the coupling of FIG. 2.

FIG. 4 is a perspective view of a suture anchor and sutures according to an example of the present application.

FIG. 5 is a perspective view of a suture anchor and sutures according to another example of the present application.

FIG. 6 is a cross-sectional view of the coupling according to the example of FIGS. 2 and 3 having the suture anchor of FIG. 5 disposed therein, according to an example of the present application.

FIG. 7 is a perspective view of a suture anchor and sutures according to another example of the present application.

FIG. 8 is a cross-sectional view of the coupling having the suture anchor of FIG. 7 disposed therein according to an example of the present application.

FIG. 9 is a perspective view of an implantable device that can be used in a reverse total shoulder arthroplasty implant according to another example of the present application.

### DETAILED DESCRIPTION

The present application relates to devices, systems, and methods that allow a suture anchor to be retained in an implantable device. In one example, the device can comprise a bone implant component configured to be secured to at least one bone of a patient. The bone implant component can have at least one recess therein. The bone implant component can be configured to allow a suture anchor to be disposed within the recess and can have a restriction therein that is engageable by the suture anchor to retain the suture anchor within the bone implant component. In a further example, the bone implant component can comprise a humeral component for use in a shoulder arthroplasty. Other examples not specifically discussed herein with reference to the FIGURES can be used with other types of devices and/or systems such as orthopedic devices, trauma devices, oncology and/or reconstructive systems.

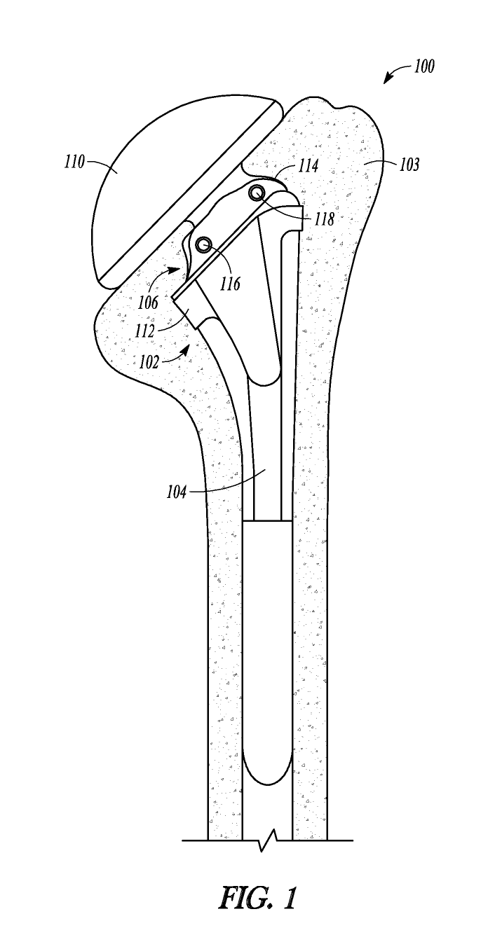

FIG. 1 shows an implantable device 100 that can comprise a humeral component 102 disposed within a humerus 103. The implantable device 100 can be for a total shoulder arthroplasty which typically includes the humeral component 102 having a stem which fits within the humeral canal, and an articulating head which articulates within the socket of a glenoid component (not shown) implanted within the glenoid of the scapula. In other instances, the implantable device 100 can be for the replacement of only the humeral component of the shoulder joint, i.e., a hemi shoulder arthroplasty, which typically includes only a humeral component that articulates within the natural glenoid socket of the scapula. As will be discussed subsequently, further examples can be used for a reverse total shoulder arthroplasty where a concave recessed articulating component is provided at the proximal end of the humeral component. This component articulates with a convex portion of the glenoid component.

The humeral component 102 can comprise a stem 104, a coupling 106, and a head 110. The stem 104 can be configured to be secured to at least one bone of a patient such as within the humeral canal of bone 103. As illustrated in FIG. 1, the stem 104 and coupling 106 can be received in the prepared canal of the humerus 103 and can be inserted therein by surgical instruments such as impactor, etc. Prior to insertion of the stem 104 and coupling 106, the proximal end of the humerus 103 can be resected and the humeral canal and proximal humeral end can be prepared using known instruments (reamers, rasps, etc.) and methods. The head 110 can seat against the resected surface of the humerus 103 and simulates the removed head.

In the example of FIG. 1, the stem 104 can have a proximal end 112 that is adapted to receive a portion of the coupling 106 therein. The coupling 106 can be connected to the stem 104 by known methods such as press-fit, fasteners, cement, adhesives, etc. In other examples, the coupling 106 can be formed as an integral part of the stem 104. A portion of the coupling 106 comprising a proximal joint portion 114 can extend from the stem 104 toward the head 110. In the example of FIG. 1, recesses comprising a medial hole 116 and a lateral hole 118, can be disposed between the head 110 and the stem 104 and are formed in the proximal joint portion 114 of the coupling 106. In other examples, the recesses can comprise slots, depressions, grooves, undercuts, etc. The recesses can be suitable to receive a suture anchor and sutures therein and retain the suture anchor as described in further detail subsequently. Although described as separate components coupling 106 can be part of the stem 104, or another component in some instances.

The coupling 106 that forms the medial hole 116 and/or the lateral hole 118 can be configured to allow a suture anchor to be disposed within the coupling 106 rather than be anchored in a bone such as the humerus 103. In particular, the medial hole 116 and/or the lateral hole 118 can have a restriction therealong that is engageable by the suture anchor to retain the suture anchor within the coupling 106. From the medial hole 116 and/or the lateral hole 118, sutures can be threaded to aid a surgeon to reconstruct the proximal humerus in the event of humeral fractures, or for the attachment of soft tissue. For example, the medial hole 116 and/or the lateral hole 118 can be used to anchor sutures wrapped around bone fragments of the upper humerus to bring the lesser and greater tuberosities into reduction circumferentially about the humeral stem, or to attach soft tissue circumferentially about humeral stem.

In some instances, the stem 104 and other components can be coated or otherwise comprised of a highly porous biomaterial useful as a bone substitute and/or cell and tissue receptive material for promotion of bone ingrowth to aid in the osseointegration of the humeral stem within the humerus. An example of such a material is produced using Trabecular Metal™ technology available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, the disclosure of which is incorporated herein by reference. As would be apparent to one skilled in the art, although the embodiments described herein utilize porous tantalum, other metals such as niobium, or alloys of tantalum and niobium with one another or with other metals may also be used.

Generally, the porous structure can include a large plurality of ligaments defining open spaces there between, with each ligament generally including a carbon core covered by a thin film of metal, such as tantalum, for example. The open spaces between the ligaments can form a matrix of continuous channels, such as having no dead ends, such as to permit uninhibited growth of cancellous bone through the porous tantalum structure. The porous structure can include up to 75%-85% or more void space therein. In an example, a porous tantalum structure can provide a lightweight, strong porous structure that can be substantially uniform and consistent in composition, and that can closely resemble the structure of natural cancellous bone, which can thereby provide a matrix into which cancellous bone can grow. The porous tantalum structure can be made in a density selected from a variety of densities, such as to selectively tailor the structure for a particular application. The porous tantalum can be fabricated to permit selecting virtually any desired porosity and pore size, and can thus be matched with the surrounding natural bone, such as to provide an improved matrix for bone ingrowth and mineralization.

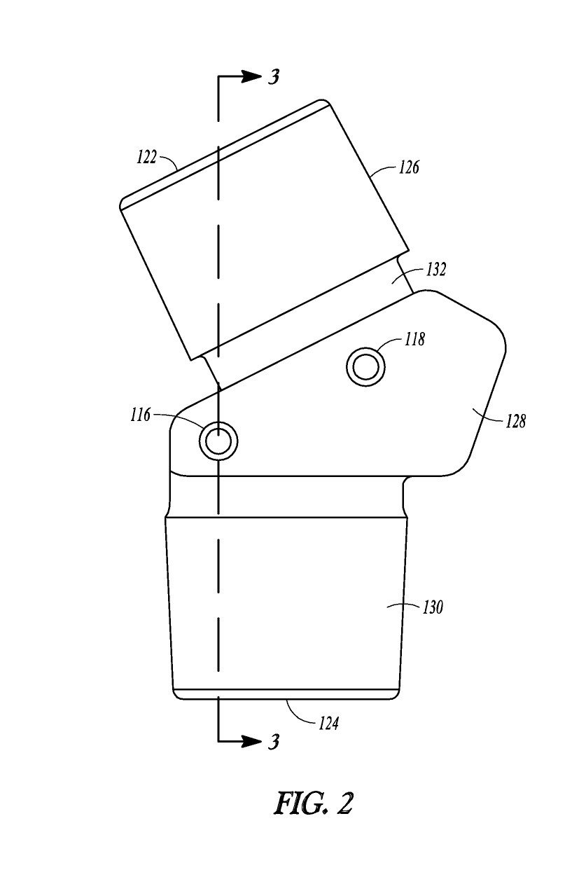

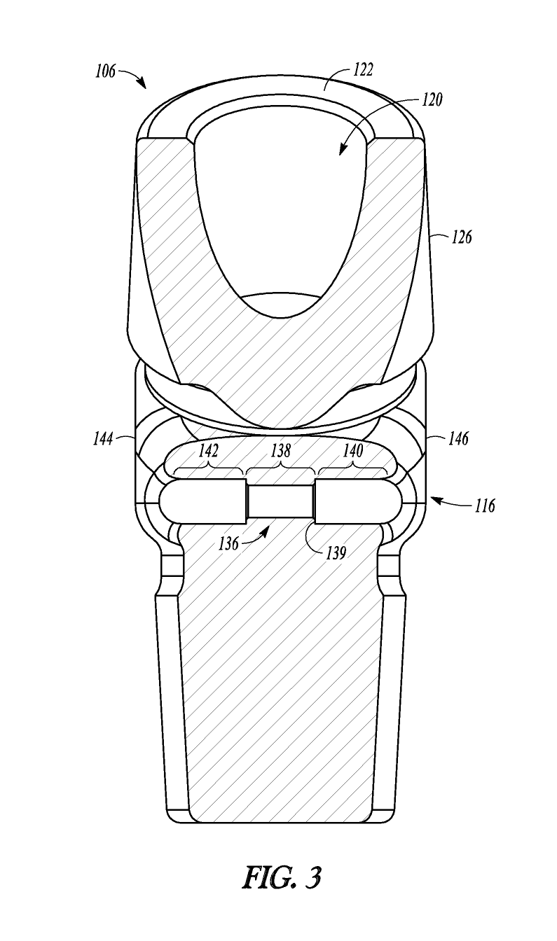

FIG. 2 shows a plan view of the coupling 106 according to one example embodiment. FIG. 3 is a cross-sectional view thorough portions of the coupling of FIG. 2. In addition to the medial and lateral holes 116 and 118, the coupling 106 can include a proximal end 122, a distal end 124, a first portion 126, a second portion 128, and a third portion 130.

The proximal end 122 of the coupling 106 can abut or be disposed adjacent the head 110 (FIGS. 1 and 2). In some instances, the proximal end 122 can be disposed within a receptacle of the head 110 or along a distal surface thereof. The distal end 124 is opposed to the proximal end 122 and can be configured for insertion into the stem 104 (FIGS. 1 and 2). The first portion 126 can be arranged at the proximal end 122 and can be formed integrally with the second portion 128. As illustrated in FIG. 2, a transition, comprising a first necked region 132 can be arranged between the first portion 126 and the second portion 128.

The second portion 128 can comprise an enlarged medial/lateral section that is partially disposed in the stem 104 (FIG. 1) and partially extends therefrom. In the example of FIG. 2, the second portion 128 can define the medial and lateral holes 116 and 118, which extend therethrough. The second portion 128 can be formed integrally with the third portion 130. The third portion 130 can be arranged at the distal end 124 of the coupling 106. A transition, comprising a second necked region 134, can be arranged between the second portion 128 and the third portion 130.

As shown in FIG. 3, the recess 120 can be defined by the first portion 126 and have an opening at the proximal end 122. The cross-sectional view of FIG. 3 extends through the medial hole 116 and illustrates a restriction 136 that can be located therein. The restriction 136 can be engageable by a suture anchor to retain the suture anchor within the coupling 106. As will be discussed subsequently, the restriction 136 can be configured (e.g., sized, shaped, have surface features, etc.) to be engaged by and retain the suture anchor within the coupling 106.

In the example shown in FIG. 3, the coupling 106 has a bi-symmetrical configuration. Thus, the coupling 106 that forms the medial hole 116 can include a first section 138, a second section 140, and a third section 142. The first section 138 can have a first diameter and the second section 140 can have an enlarged diameter with respect to the first diameter of the first section 138. The first section 138 can include a transition surface 139 disposed between the first section 138 and the second section 140. In some cases the third section 142 can have substantially a same diameter as the second section 140. The medial hole 116 can extend through the coupling 106 such that the first section 138 can be accessed from either a first side 144 or second side 146 of the coupling 106.

The first section 138 can comprise a suture anchor retention feature for the coupling 106. Additionally, the second section 140 and/or third section 142 can act as a suture anchor and suture access feature to an interior of the coupling 106. In some instances, the first section 138 can act as the restriction 136 to retain the suture anchor as will be discussed in further detail subsequently.

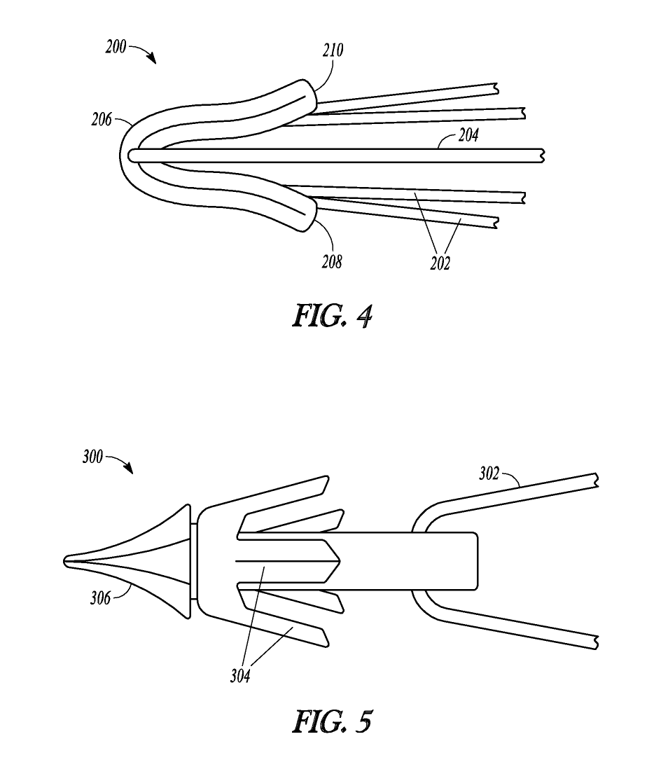

FIGS. 4 and 5 illustrate two examples of suture anchors 200 and 300 and associated sutures 202 and 302 that can be used with implantable devices such as implantable device 100 (FIG. 1). FIG. 4 illustrates anchor 200 which can have a soft sleeve braided construction. An example of such an anchor is the JuggerKnot™ Soft Anchor, which is commercially available and manufactured by Biomet, Inc. of Warsaw, Ind. Suture anchors such as the anchor 200 are also disclosed in U.S. Patent Appl. Pub. Nos. 2014/0052178A1 and 20140052179A1, the disclosures of which are incorporated herein by reference. The suture anchor 200 can be delivered into the implantable device recess by a surgical instrument 204 such as an inserter or similar pusher instrument. The suture 202 can comprise FiberWire® or TigerWire® flexible strand, for example. The suture 202 can be threaded through a sheath 206 of the suture anchor 200 so as to form a loop. The sheath 206 can have sufficient resiliency such that first and second ends 208 and 210 of the loop are positionable outward and away from one another (e.g. toward walls of a recess).

The suture anchor 300 of FIG. 5 can have a number of prongs 304 extending away from a head 306 thereof. An example of a similar suture anchor is the SuperAnchor™, which is commercially available and manufactured by Ethicon, Inc. of Westwood, Mass. In some cases, the prongs 304 can be shaped to allow the suture anchor 300 to be “set” within a recess by a pull-out force that brings the prongs 304 into contact with one or more walls thereof.

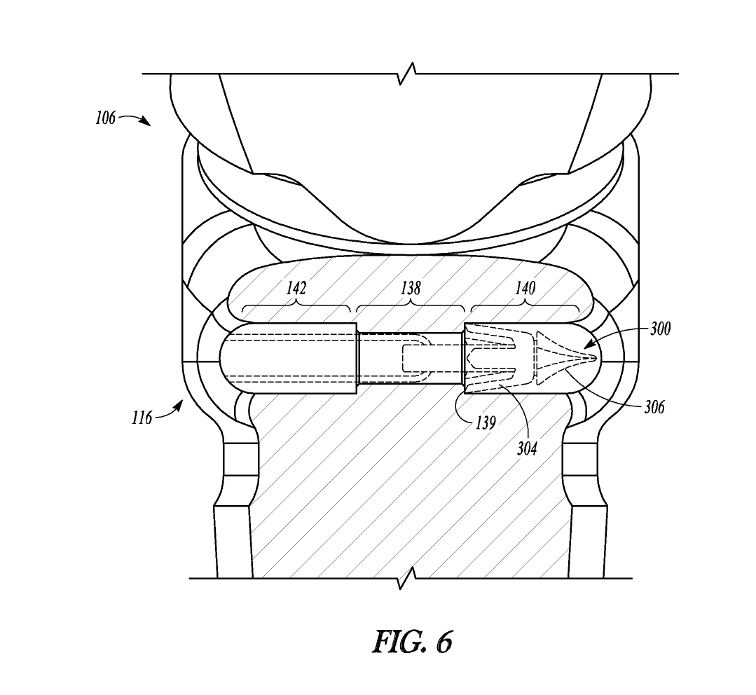

FIG. 6 illustrates the suture anchor 300 can be inserted into the coupling 106. The suture anchor 300 can be positioned within the recess (e.g., medial opening 116) and actuated (set, placed, threaded, etc.) to engage a wall (e.g., transition surface 139, etc.) of the recess to secure the suture anchor 300 within the coupling 106. In particular, the suture anchor 300 can be inserted through the first section 138 (and the third section 142) to the second section 140 in a temporary shape. In the case of the suture anchor 300, the temporary shape can include instances where the prongs 304 of the suture anchor 300 are be compressed by the diameter of the first section 138. In the case of suture anchor 200 (FIG. 4), the temporary shape can include instances where the force of an insertion tool on a midsection of the anchor causes a change in shape. Additionally or alternatively, the temporary shape of the suture anchor 200 can include instances where the ends 208 and 210 (FIG. 4) are resolved toward one another by the diameter of the first section 138.

Upon insertion into the second section 140, the suture anchor 300 can change shape from the temporary delivery shape to an engagement shape as illustrated in FIG. 6. In the engagement shape, the prongs 304 are biased such that they expand outward from one another and the head 306. In some instances, the prongs 304 can make contact with a diametrical wall of the second section 140. In a similar manner, the ends 208 and 210 of suture anchor 200 (FIG. 4) can be biased away from one another so as to expand outward and in some instances make contact with the diametrical wall and/or transition surface 139.

In some instances, the suture anchor 300 can be “set” with a withdrawal force applied to the suture 302 such that the suture anchor 300 is brought into contact with at least a portion of the first section 138 such as the transition surface 139. Similarly, the ends 208 and 210 of suture anchor 200 (FIG. 4) can be brought into contact with at least a portion of the first section 138 such as the transition surface 139. Thus, the transition surface 139 can act as a shoulder to retain the suture anchor against the first section 138 and substantially within the second section 140 of the coupling 106. In other instances, suture anchors could diametrically engage the first section 138 rather than being inserted into the second section 140. Thus, in some cases the suture anchor could be disposed substantially within only the first section 138.

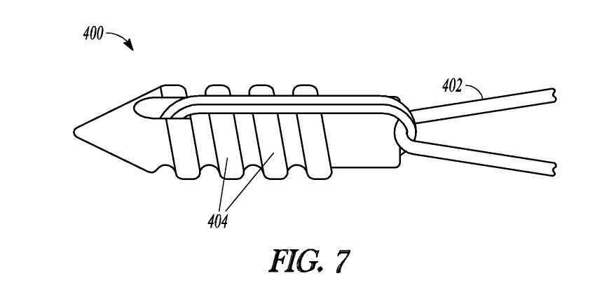

FIG. 7 illustrates another example of a suture anchor 400 and associated sutures 402 that can be used with implantable devices such as implantable device 100 (FIG. 1). The suture anchor 400 includes a thread 404 along an exterior thereof. Examples of such a suture anchor include the Statak™ Soft Tissue Attachment Device and the Bio-Statak® Soft Tissue Attachment Device, which are commercially available and manufactured by Zimmer, Inc. of Warsaw, Ind.

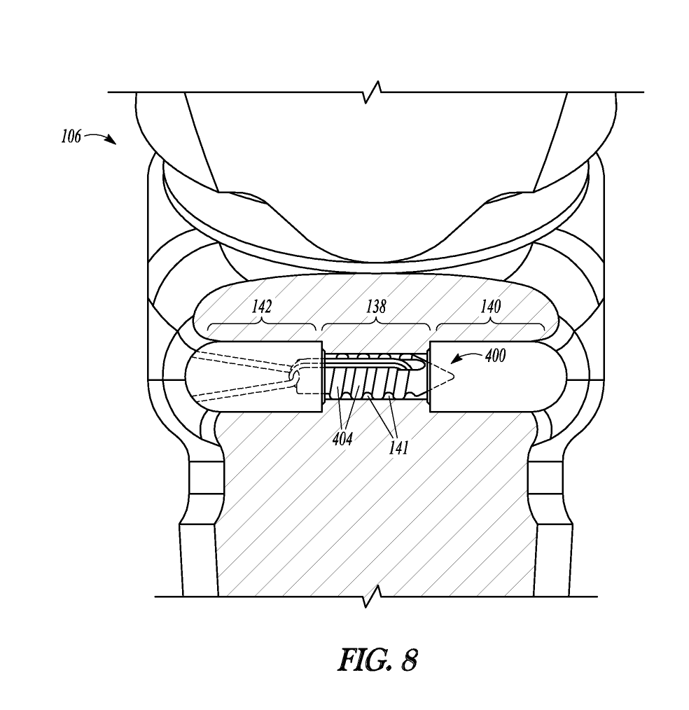

FIG. 8 illustrates the suture anchor 400 inserted into the coupling 106. The suture anchor 400 can be positioned within the recess (e.g., medial opening 116) and actuated to engage a wall (e.g., diametrical surface 141, etc.) of the recess to secure the suture anchor 400 within the coupling 106. In particular, the suture anchor 300 can be inserted into the first section 138 through the third section 142. The diametrical surface 141 can have a thread form configured to mate with the thread form 404 of the suture anchor 400. Thus, actuating the suture anchor 400 in some cases can comprise treading the suture anchor 400 into the mating thread in the diametrical wall 141 that defines the recess. In the example of FIG. 8, the suture anchor 400 can be disposed substantially within only the first section 138.

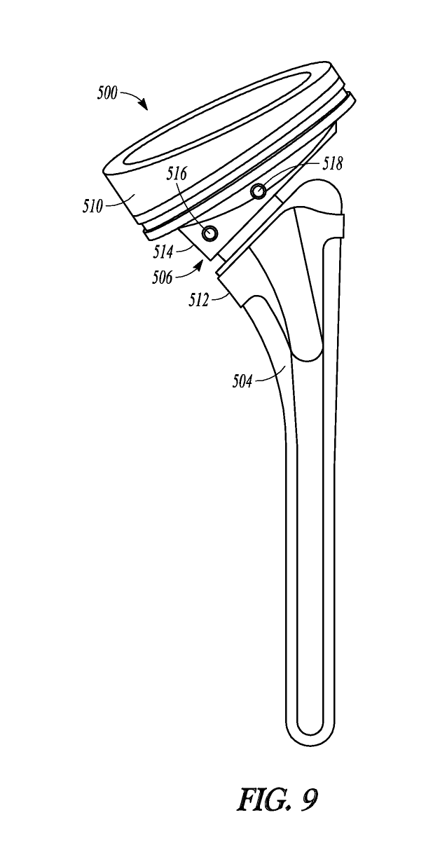

FIG. 9 illustrates an implantable device 500 that comprises a humeral component 502 that can be disposed within a humerus such as humerus 103 (FIG. 1) in a similar manner to implantable device 100. The implantable device 500 can be used in a reverse total shoulder arthroplasty. A reverse type implantable device, such as implantable device 500 utilizes a concave recessed articulating component provided at the proximal end of the humeral component 502 that articulates against a convex portion of a glenoid component (not shown).

The humeral component 502 can comprise a stem 504, a coupling 506, and an articular surface 510. The stem 504 can be configured to be secured to at least one bone of a patient such as within the humeral canal of the humerus. The stem 504 can be placed in the humeral canal in a manner similar the manner described in reference to FIG. 1. A portion of the coupling 506 can be designed to seat against the resected surface of the humerus and simulates the removed head but in the reverse fashion previously described. The articular surface 510 can seat against a glenosphere component (not shown) anchored to the patient's scapula.

In FIG. 9, the stem 504 can have a proximal end 512 that is adapted to receive a portion of the coupling 506 therein. The coupling 506 can be connected to the stem 504 by known methods such as press-fit, fasteners, cement, adhesives, etc. In other examples, the coupling 106 can be formed as an integral part of the stem 504 rather than as a separate component. A portion of the coupling 506 that comprises a proximal joint portion 514 can be disposed adjacent the articular surface 510. In the example of FIG. 9, the recesses comprise a medial hole 516 and a lateral hole 518, which can be disposed between the articular surface 510 and the stem 504 and are formed in the proximal joint portion 514 of the coupling 506. In other examples, the recesses can comprise slots, depressions, grooves, undercuts, etc. These recesses are suitable to receive a suture anchor therein and retain the suture anchor as described previously with reference to various examples.

Although specific configurations of an implantable device and suture anchors are shown in FIGS. 1-9 and particularly described above, other designs of an implantable device and suture anchors that fall within the scope of the claims are anticipated. For example, the implantable device with suture anchor fixation capability can be extended to orthopedic devices for artificial knees, ankles, elbows, and hips. Additionally, the implantable device with suture anchor fixation capability can be utilized for trauma devices, oncology and/or reconstructive systems.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An implantable device comprising:
a bone implant component configured to be secured to at least one bone of a patient and having at least one recess therein, wherein the bone implant component is configured to allow a suture anchor to be disposed within the recess and has a restriction therein that is engageable by the suture anchor to retain the suture anchor within the bone implant component.

2. The device of claim 1 wherein the recess comprises a hole formed by a first section with a first diameter and a second section having an enlarged diameter with respect to the first diameter of the first section, and wherein the first section acts as the restriction to retain the suture anchor.

3. The device of claim 2 wherein the first section includes a transition surface between the first section and the second section, the transition surface acts as a shoulder to retain the suture anchor against the first section and substantially within the second section of the bone implant component.

4. The device of claim 2 wherein the hole extends through the bone implant component such that the first section can be accessed from either a first or second side of the bone implant component.

5. The device of claim 2 wherein the bone implant component has a bi-symmetrical configuration such that the hole includes the first section, the second section, and a third section, and wherein the third section has a diameter that is substantially the same as the diameter of the second section.

6. The device of claim 2 wherein the first section comprises a suture anchor retention feature and the second section comprises a suture anchor access feature.

7. The device of claim 2 wherein a wall of the first section has a thread configured to mate with a thread of the suture anchor.

8. The device of claim 1 wherein the bone implant component comprises a humeral component for use in a shoulder arthroplasty.

9. The device of claim 8 wherein the bone implant component further comprises a coupling of the humeral component that includes the at least one recess.

10. A surgical method comprising:
securing a bone implant component to at least one bone of a patient, the bone implant component having at least one recess therein;
positioning a suture anchor within the recess; and
actuating the suture anchor to engage a wall of the recess to secure the suture anchor within the bone implant component.

11. The method of claim 10 wherein actuating the suture anchor comprises:
inserting the suture anchor in a temporary shape through a first section to a second section of the recess, wherein the first section has a reduced diameter with respect to a diameter of the second section; and
bringing the suture anchor into contact with at least a portion of the first section.

12. The method of claim 11 wherein the portion of the first section comprises a transition surface between the first section and the second section, the transition surface acts as a shoulder to retain the suture anchor against the first section and substantially within the second section of the bone implant component.

13. The method of claim 10 wherein actuating the suture anchor comprises threading the suture anchor into a mating thread in the wall of the recess.

14. The method of claim 10 wherein the bone implant component comprises a humeral component for use in a shoulder arthroplasty.

15. A system comprising:
a shoulder implant having a humeral component configured to be secured to at least one bone of a patient and having at least one recess with a restriction therealong; and
a suture anchor engageable with the restriction to retain a suture anchor within the humeral component.

16. The system of claim 15 wherein the recess comprises a hole formed by a first section with a first diameter and a second section having an enlarged diameter with respect to the first diameter of the first section, and wherein the first section comprises the restriction.

17. The system of claim 16 wherein the first section includes a transition surface between the first section and the second section, the transition surface acts as a shoulder to retain the suture anchor against the first section and substantially within the second section of the humeral component.

18. The system of claim 16 wherein the hole extends through the humeral component such that the first section can be accessed from either a first or second side of the humeral component.

19. The system of claim 16 wherein the humeral component has a bi-symmetrical configuration comprising the first section, the second section, and a third section, and wherein the third section has a diameter that is substantially the same as the diameter of the second section.

20. The system of claim 15 wherein a wall of the restriction has a thread configured to mate with a thread of the suture anchor.
